(19) United States
(12) Patent Application Publication
(10) Pub. No.: US 2008/0306098 A1
(43) Pub. Date: Dec. 11, 2008

(54) Pharmacokinetics of protease inhibitors and other drugs

(76) Inventors: Mitchell W. Mutz, La Jolla, CA (US); Jason E. Gestwicki, Ann Arbor, MI (US)

Correspondence Address: MINTZ, LEVIN, COHN, FERRIS, GLOVSKY AND POPEO, P.C, 5 Palo Alto Square - 6th Floor, 3000 El Camino Real, PALO ALTO, CA, 94306-2155, US

(21) Appl. No.: 12/151,329

(22) Filed: May 5, 2008

(30) Foreign Application Priority Data
Nov. 6, 2006 (US) PCT/US2006/043400

(51) Int. Cl. A61K 31/505; A61K 31/4353; C07D 491/12; C07D 239/04; A61P 31/18

(52) U.S. Cl. 514/274; 514/291; 546/90; 544/316

(57) ABSTRACT

A method for modulating at least one pharmacokinetic property of a protease inhibitor upon administration to a host is provided. One administers to the host an effective amount of a bifunctional compound of less than about 5000 daltons comprising the protease inhibitor or an active derivative thereof and a pharmacokinetic modulating moiety. The pharmacokinetic modulating moiety binds to at least one intracellular protein. The bifunctional compound has at least one modulated pharmacokinetic property upon administration to the host as compared to a free drug control that comprises the protease inhibitor.

### CROSS-REFERENCE TO RELATED APPLICATIONS

This application claims priority to PCT/US2006/043400 filed Nov. 6, 2006, which claims priority to U.S. provisional application Ser. No. 60/734,197, filed Nov. 5, 2005. Both priority documents are incorporated by reference in their entireties.

### TECHNICAL FIELD

This invention relates generally to pharmacology and more specifically to the modification of known active agents to give them more desirable properties.

### BACKGROUND ART

When HIV was first discovered, it was feared that all persons infected with HIV would eventually develop full-blown AIDS. However, drugs were developed and approved which could slow the proliferation of the HIV virus. The most usual therapies over the last decade for HIV-infected people have been three-drug cocktails, in which one of the drugs is an HIV protease inhibitor and the other two are reverse-transcriptase inhibitors. The introduction of three-drug cocktails in the mid-1990s has allowed many HIV-infected people to survive for long periods of time without developing AIDS and has allowed some AIDS patients to experience a notable remission.

Patient compliance and drug toxicity have always been major issues with HIV protease inhibitors due to the frequency of dosage and co-administration of other HIV therapeutics. A reduction in dosage frequency would represent an improvement in quality of life for the patient and a lower chance of toxic side effects due to decreased production of secondary metabolites, especially in the liver. Although recent HIV protease inhibitors require a reduced dosage burden compared with earlier drugs, there is still significant opportunity for improving PIs by reducing first pass clearance via cytochrome P450 enzymes and increasing the drug half-life in the circulation.

Examples of HIV protease inhibitors which have diminished half lives due to poor pharmacokinetics are amprenavir, lopinavir, indinavir and ritonavir, among others.

Previous methods to improve pharmacokinetics (PK) of HIV protease inhibitors include: medicinal chemistry-based analog synthesis, employing pro-drug strategies, improved formulation, and co-administration with P450 and P-glycoprotein inhibitors. Regardless of the methods employed, these approaches share a desired outcome: improve pharmacokinetics to make treatment easier for patients. However, these methods have not yielded an inhibitor that requires a substantially lower dose (e.g., once or twice per week) over the prior inhibitors and is relatively non-toxic compared with prior inhibitors.

There is therefore still a need in the art for protease inhibitors and associated dosage forms which have reduced first pass clearance and/or improved pharmacokinetics.

### DISCLOSURE OF THE INVENTION

In an embodiment of this invention, a method for modulating at least one pharmacokinetic property of a protease inhibitor upon administration to a host is provided. One administers to the host an effective amount of a bifunctional compound of less than about 5000 daltons comprising the protease inhibitor or an active derivative thereof and a pharmacokinetic modulating moiety. The pharmacokinetic modulating moiety binds to at least one intracellular protein. The bifunctional compound has at least one modulated pharmacokinetic property upon administration to the host as compared to a free drug control that comprises the protease inhibitor.

In a further embodiment of this invention, a bifunctional compound comprising protease inhibitor functionality and a pharmacokinetic modulating moiety are provided.

In a further aspect of the invention, a bifunctional compound is provided in a pharmaceutical formulation which is designed to have a controlled release mechanism in addition to that provided by the bifunctional compound. The bifunctional compound may comprise a drug moiety that is a protease inhibitor or some other drug.

### BRIEF DESCRIPTION OF THE DRAWINGS

FIG. 1A depicts the structure of FK506 linked to a modular linker and target binding moiety, for example a protease inhibitor. Due to the modular nature of the synthesis, the linker group and target-binding group have been altered. FIG. 1B illustrates how the steric bulk of an FKBP protein can confer protection from P450 enzymes.

In FIG. 2, the left side depicts the bimodal binding character of FK506 whereby it binds both FKBP and calcineurin. The schematic on the right depicts how the calcineurin-binding mode can be eliminated by substituting a linker and target binding moiety. In this manner, FK506 can simultaneously target FKBP and bind a second protein.

In FIG. 3, (A) shows the structure of FK506 bound to curcumin. (B) illustrates that FK506-curcumin is protected from CYP3a4, a P450 enzyme, in the presence of FKBP. (C) gives a schematic of the Invitrogen assay used.

In FIG. 4, the left side illustrates an exemplary synthetic scheme for bifunctional compounds of the invention. The right side shows the application of this scheme to amprenavir, lopinavir, and ritonavir.

FIG. 5 depicts useful linkers which have amine and (alkyl-protected) carboxyl moieties.

FIG. 6 sets out a synthetic scheme for the synthesis of an amprenavir-based SLF-PI conjugate. The amprenavir-like moiety is shown at the right in FIG. 6.

FIGS. 7A-7B depict a reaction schema for ritonavir and lopinavir respectively. Note the schema proceeds via analogous chemistry using the well-characterized Boc (t-butoxycarbonyl) leaving group.

### DETAILED DESCRIPTION OF PREFERRED EMBODIMENTS

Before describing the present invention in detail, it is to be understood that this invention is not limited to specific solvents, materials, or device structures, as such may vary. It is also to be understood that the terminology used herein is for the purpose of describing particular embodiments only, and is not intended to be limiting.

As used in this specification and the appended claims, the singular forms “a,” “an,” and “the” include both singular and plural referents unless the context clearly dictates otherwise. Thus, for example, reference to “an active ingredient” includes a plurality of active ingredients as well as a single active ingredient, reference to “a temperature” includes a plurality of temperatures as well as single temperature, and the like.

The term “bifunctional compound” refers to a non-naturally occurring compound that includes a pharmacokinetic modulating moiety and a drug moiety, where these two components may be covalently bonded to each other either directly or through a linking group. The term “drug” refers to any active agent that affects any biological process. Bifunctional compounds may have more than two functionalities.

The pharmacokinetic modulating moiety may be a peptide or protein and may also be an enzyme or nucleic acid. Similarly, the drug moiety may also be peptide, protein, enzyme, or nucleic acid.

Active agents which are considered drugs for purposes of this application are agents that exhibit a pharmacological activity. Examples of drugs include active agents that are used in the prevention, diagnosis, alleviation, treatment or cure of a disease condition.

By “pharmacologic activity” is meant an activity that modulates or alters a biological process so as to result in a phenotypic change, e.g. cell death, cell proliferation etc.

By “pharmacokinetic property” is meant a parameter the describes the disposition of an active agent in an organism or host. Representative pharmacokinetic properties include: drug half-life, hepatic first-pass metabolism, volume of distribution, degree of blood serum protein, e.g. albumin, binding, etc, degree of tissue targeting, cell type targeting.

By “half-life” is meant the time for one-half of an administered drug to be eliminated through biological processes, e.g. metabolism, excretion, etc.

By “hepatic first-pass metabolism” is meant the propensity of a drug to be metabolized upon first contact with the liver, i.e. during its first pass through the liver.

By “volume of distribution” is meant the distribution and degree of retention of a drug throughout the various compartments of an organisms, e.g. intracellular and extracellular spaces, tissues and organs, etc.

The term “efficacy” refers to the effectiveness of a particular active agent for its intended purpose, i.e. the ability of a given active agent to cause its desired pharmacologic effect.

The term “host” refers to any mammal or mammalian cell culture or any bacterial culture.

Where the term HIV is used, it is understood that the invention may be employed on relative protease inhibitors such as found in other immunodeficiency viruses found in non-human species or human variants (HIV II, SIV, FIV, etc).

Where FK506 is used, variants or analogs of FK506 are included, such as rapamycin, pimecrolimus, or synthetic ligands of FK506 binding proteins (SLFs) such as those disclosed in U.S. Pat. Nos. 5,665,774, 5,622,970, 5,516,797, 5,614,547, and 5,403,833 or described by Holt et al., “Structure-Activity Studies of Synthetic FKBP Ligands as Peptidyl-Prolyl Isomerase Inhibitors,” Bioorganic and Medicinal Chemistry Letters, 4(2):315-320 (1994).

In an embodiment of this invention, a method for modulating at least one pharmacokinetic property of a protease inhibitor upon administration to a host is provided. One administers to the host an effective amount of a bifunctional compound of less than about 5000 daltons comprising the protease inhibitor or an active derivative thereof and a pharmacokinetic modulating moiety. The pharmacokinetic modulating moiety binds to at least one intracellular protein. The bifunctional compound has at least one modulated pharmacokinetic property upon administration to the host as compared to a free drug control that comprises the protease inhibitor.

Bifunctional compound in general have aroused considerable interest in recent years. See, for example, U.S. Pat. No. 6,270,957, U.S. Pat. No. 6,316,405, U.S. Pat. No. 6,372,712, U.S. Pat. No. 6,887,842, and U.S. Pat. No. 6,921,531. ConjuChem (Montreal, Canada) scientists have shown that covalent coupling of insulin to human serum albumin can improve the half-life from 8 hours to over 48 hours. Xenoport (Santa Clara, Calif.) has pioneered attachment of receptor ligands to improve drug uptake and distribution. Human trials of methotrexate-albumin conjugates revealed that the modified methotrexate had half-lives of up to two weeks compared with 6 hours for unmodified methotrexate. Other examples include PEGylation of growth factors and attachment of folate groups that “target” anti-cancer drugs. All these strategies use modification of a “parent” drug to provide new binding profiles or enhanced protection from degradation.

More recently, a team including one of the inventors attached SLF to ligands for amyloid beta. Amyloid beta oligomers are believed to underlie the neuropathology of Alzheimer's disease. Therefore, methods to decrease amyloid aggregation are of therapeutic interest. Amyloid ligands, such as congo red or curcumin (above), can be synthetically coupled to FK506 or SLF. The resulting bifunctional compound binds both FKBP and amyloid beta. These molecules are potent inhibitors of amyloid aggregation and they block neurotoxicity. See Jason E. Gestwicki et al., “Harnessing Chaperones to Generate Small-Molecule Inhibitors of Amyloid β Aggregation,” Science 306:865-69 (2004).

Bifunctional compounds of the type employed in the present invention are generally described by the formula:

X-L-Z

wherein:

X is a drug moiety;

L is a bond or linking group; and

Z is a pharmacokinetic modulating moiety,

with the proviso that X and Z are different. Thus, as may be seen, a bifunctional compound is a non-naturally occurring or synthetic compound that is a conjugate of a drug or derivative thereof and a pharmacokinetic modulating moiety, where these two moieties are optionally joined by a linking group.

In bifunctional compounds used in the invention the pharmacokinetic modulating and drug moieties may be different, such that the bifunctional compound may be viewed as a heterodimeric compound produced by the joining of two different moieties. In many embodiments, the pharmacokinetic modulating moiety and the drug moiety are chosen such that the corresponding drug target and any binding partner of the pharmacokinetic modulating moiety, e.g., a pharmacokinetic modulating protein to which the pharmacokinetic modulating moiety binds, do not naturally associate with each other to produce a biological effect.

The bifunctional compounds are typically small. As such, the molecular weight of the bifunctional compound is generally at least about 100 D, usually at least about 400 D and more-usually at least about 500 D. The molecular weight may be less than about 800 D, about 1000 D, about 1200 D, or about 1500 D, and may be as great as 2000 D or greater, but usually does not exceed about 5000 D. The preference for small molecules is based in part on the desire to facilitate oral administration of the bifunctional compound. Molecules that are orally administrable tend to be small.

The pharmacokinetic modulating moiety modulates a pharmacokinetic property, e.g. half-life, hepatic first-pass metabolism, volume of distribution, degree of albumin binding, etc., upon administration to a host as compared to free drug control. By modulated pharmacokinetic property is meant that the bifunctional compound exhibits a change with respect to at least one pharmacokinetic property as compared to a free drug control. For example, a bifunctional compound of the subject invention may exhibit a modulated, e.g. longer, half-life than its corresponding free drug control. Similarly, a bifunctional compound may exhibit a reduced propensity to be eliminated or metabolized upon its first pass through the liver as compared to a free drug control. Likewise, a given bifunctional compound may exhibit a different volume of distribution that its corresponding free drug control, e.g. a higher amount of the bifunctional compound may be found in the intracellular space as compared to a corresponding free drug control. Analogously, a given bifunctional compound may exhibit a modulated degree of albumin binding such that the drug moiety's activity is not as reduced, if at all, upon binding to albumin as compared to its corresponding free drug control. In evaluating whether a given bifunctional compound has at least one modulated pharmacokinetic property, as described above, the pharmacokinetic parameter of interest is typically assessed at a time at least 1 week, usually at least 3 days and more usually at least 1 day following administration, but preferably within about 6 hours and more preferably within about 1 hour following administration.

The linker L, if not simply a bond, may be any of a variety of moieties chosen so that they do not have an adverse effect on the desired operation of the two functionalities of the molecule and also chosen to have an appropriate length and flexibility. The linker may, for example, have the form F₁—(CH₂)ₙ—F₂ where F₁ and F₂ are suitable functionalities. A linker of this sort comprising an alkylene group of sufficient length may allow, for example, for the free rotation of the drug moiety even when the pharmacokinetic modulating moiety is bound. Alternatively, a stiffer linker with less free rotation may be desired. The hydrophobicity of the linker is also a relevant consideration. FIG. 5 depicts some precursors which may be used for the linker (with the carboxyl functionality protected).

The drug moiety X may, in certain embodiments of the invention, preferably be a protease inhibitor. The drug moiety may be derived from a known protease inhibitor, which is preferably effective against HIV and/or against another prevalent virus such as hepatitis B. The drug moiety preferably has a functionality which may readily and controllably be made to react with a linker precursor. HIV protease inhibitors include, for example, atazanavir, saquinavir, ritonavir, indinavir, nelfinavir, amprenavir, fosamprenavir, mozenavir, TMC114 (darunavir), tipranavir, and lopinavir. The known HIV protease inhibitors are generally susceptible to metabolism and subsequent deactivation by hepatic first-pass clearance mechanisms. Protease inhibition is an active area of research.

Certain of the concepts of this invention have applicability to other drug moieties besides protease inhibitors. In general, bifunctional compounds may usefully be made with any drug having a suitable moiety capable of reacting with linkers and which has a need for pharmacokinetic modulation. Thus, for example, drugs having a strong first-pass effect may be candidates for incorporation into a bifunctional compound.

In general, the pharmacokinetic modulating moiety Z will be one which is capable of reversible attachment to a common protein, meaning one which is abundant in the body or in particular compartments of the body or particular tissue types. Common proteins include, for example, FK506 binding proteins, cyclophilin, tubulin, actin, heat shock proteins, and albumin. Common proteins are present in concentrations of at least 1 micromole, preferably at least 10 micromoles, more preferably at least 100 micromoles, and even more preferably 1 millimole in the body or in particular compartments or tissue types. The pharmacokinetic modulating moiety should, like the drug, have a moiety which is capable of reacting with suitable linkers.

It is desirable for at least some embodiments of the present invention that the binding of the pharmacokinetic modulating moiety Z to a common protein be such as to sterically hinder the activity of common metabolic enzymes such as CYP450 enzymes when the bifunctional compound is so bound. Persons of skill in the art will recognize that the effectiveness of this steric hindrance depends, among other factors, on the conformation of the common protein in the vicinity of the pharmacokinetic modulating moiety's binding site on the protein, as well as on the size and flexibility of the linker. The choice of a suitable linker and pharmacokinetic modulating moiety may be made empirically or it may be made by means of molecular modeling of some sort if an adequate model of the interaction of candidate pharmacokinetic modulating moieties with the corresponding common proteins exists.

The attachment point and linker characteristics are preferably selected based on structural information such that the inhibitory potency of the protease inhibitor is preserved, giving the desired superior pharmacokinetic characteristics.

Where the pharmacokinetic modulating moiety operates by binding a protein, it may be referred to as a “presenter protein ligand” and the protein which it binds to may be referred to as a “presenter protein.”

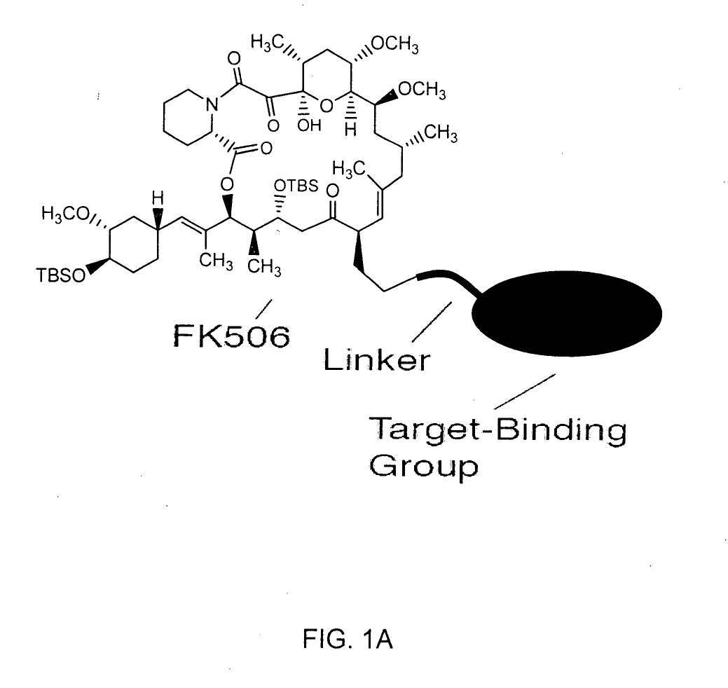

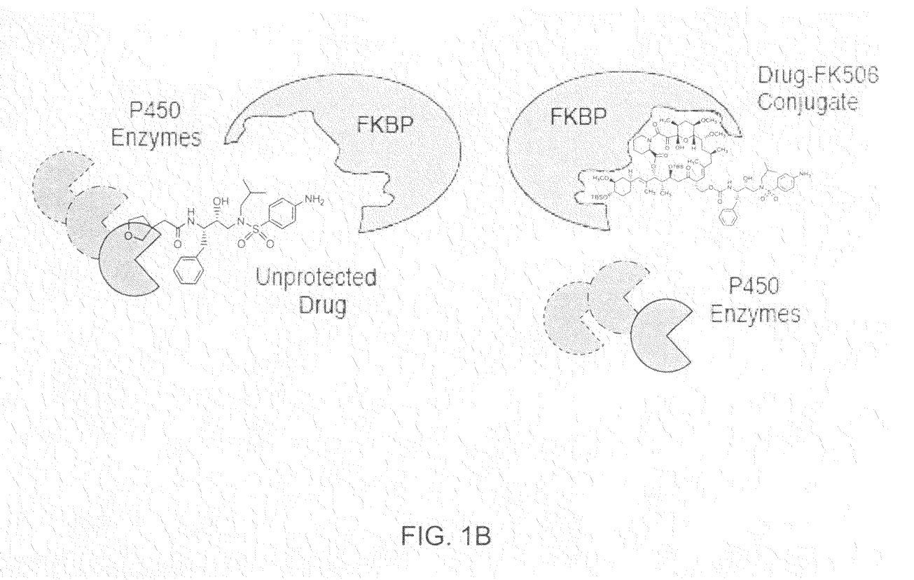

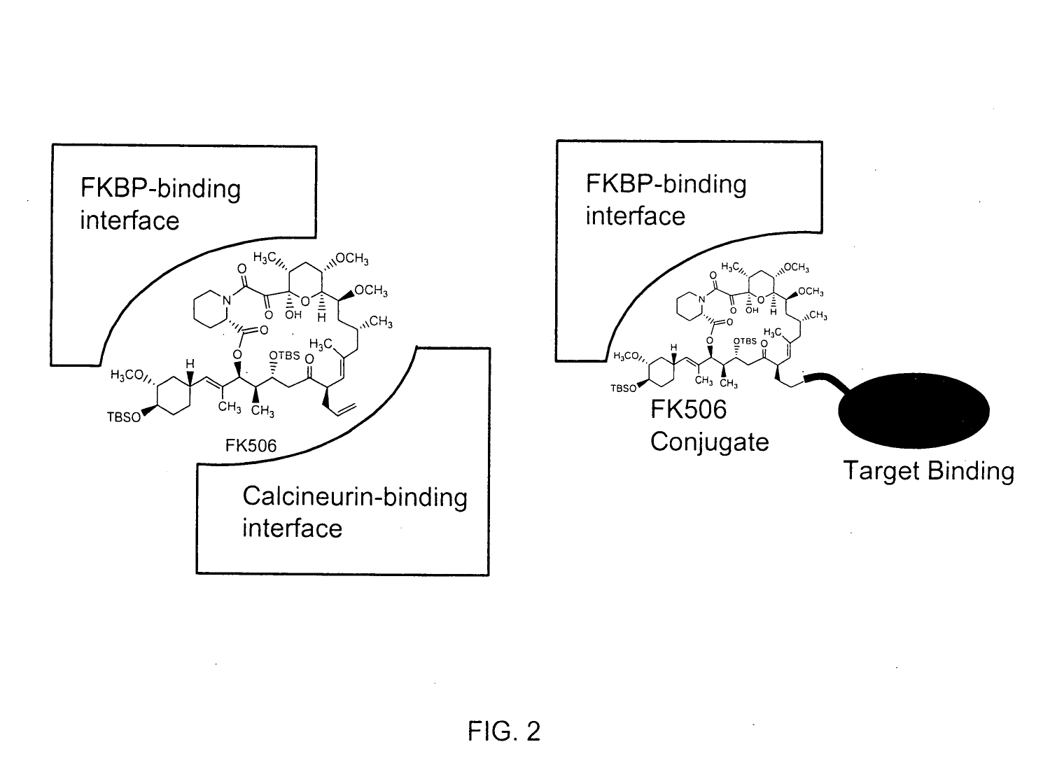

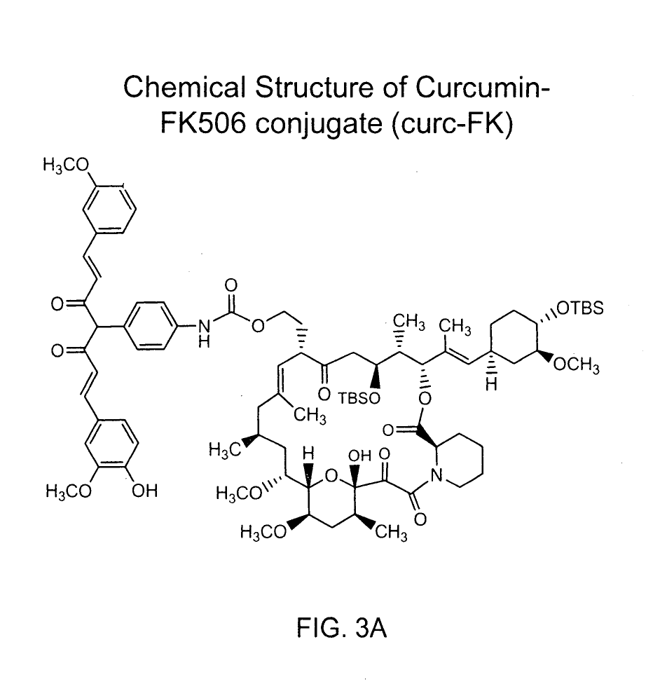

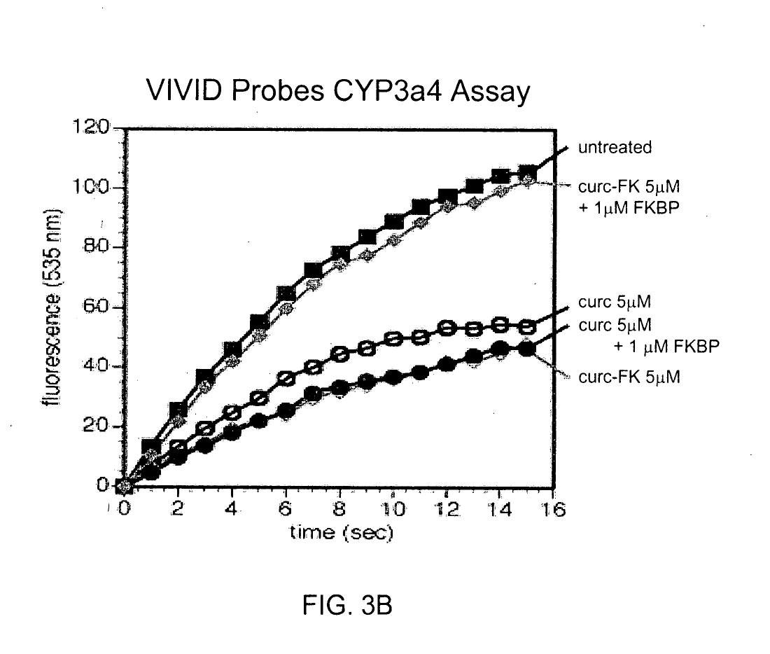

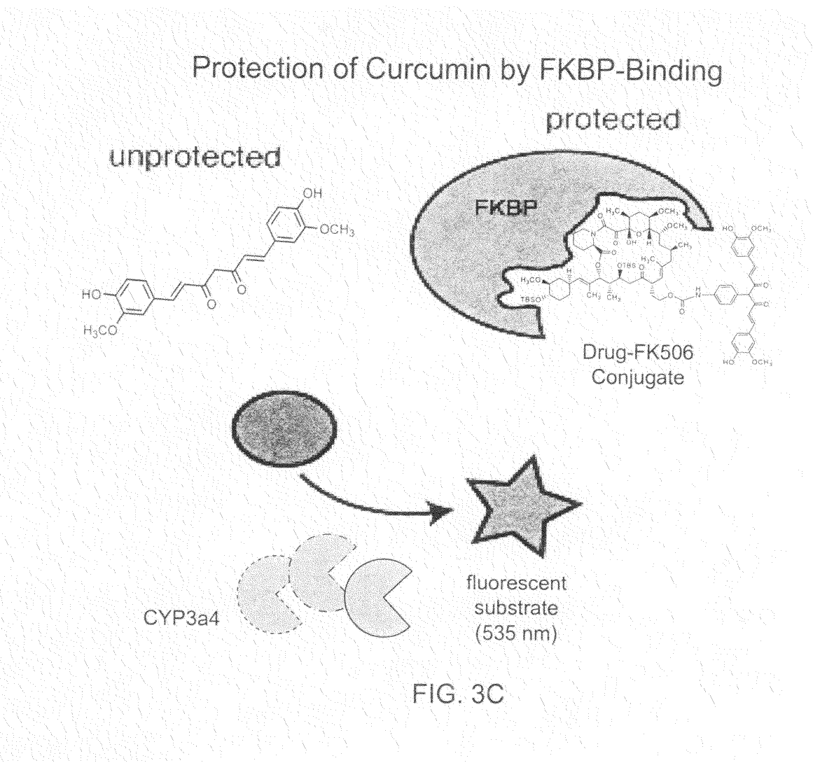

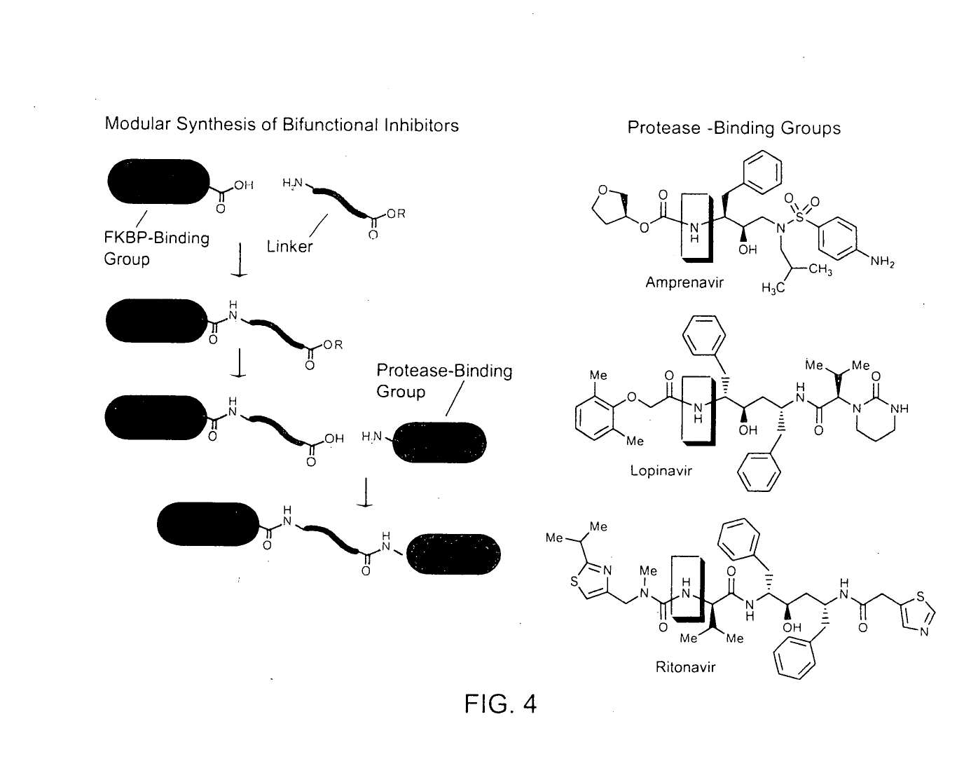

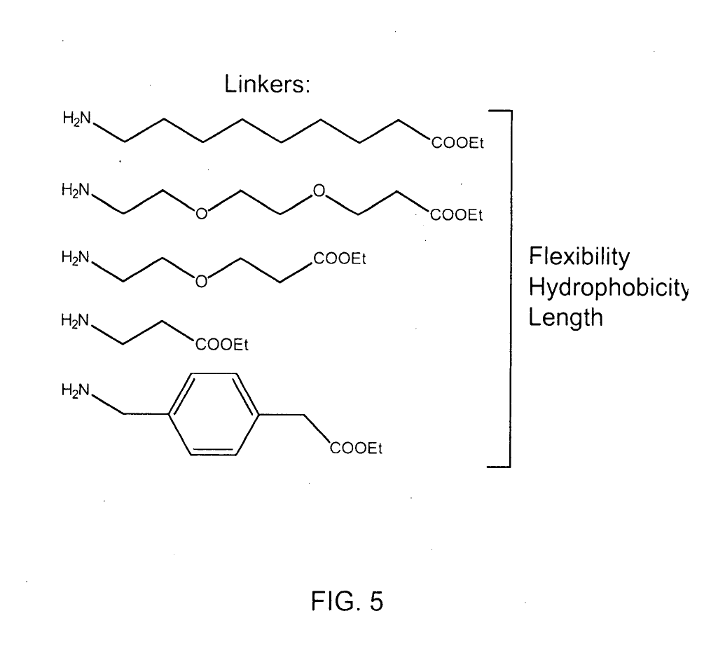

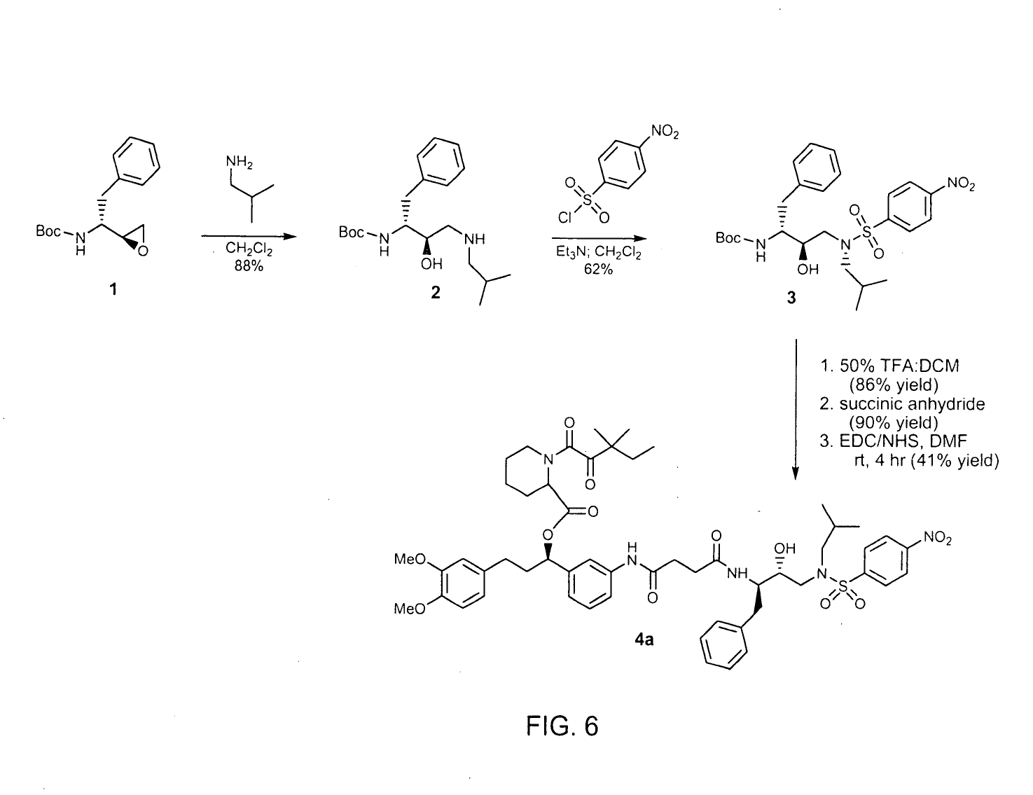

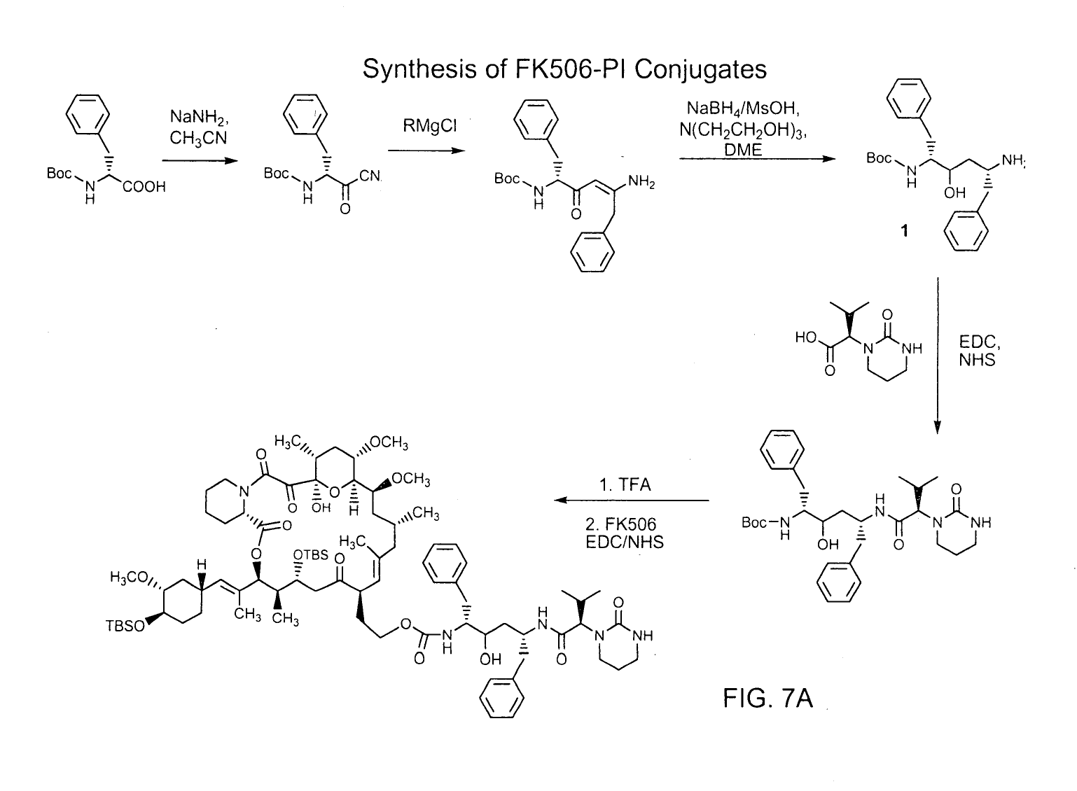

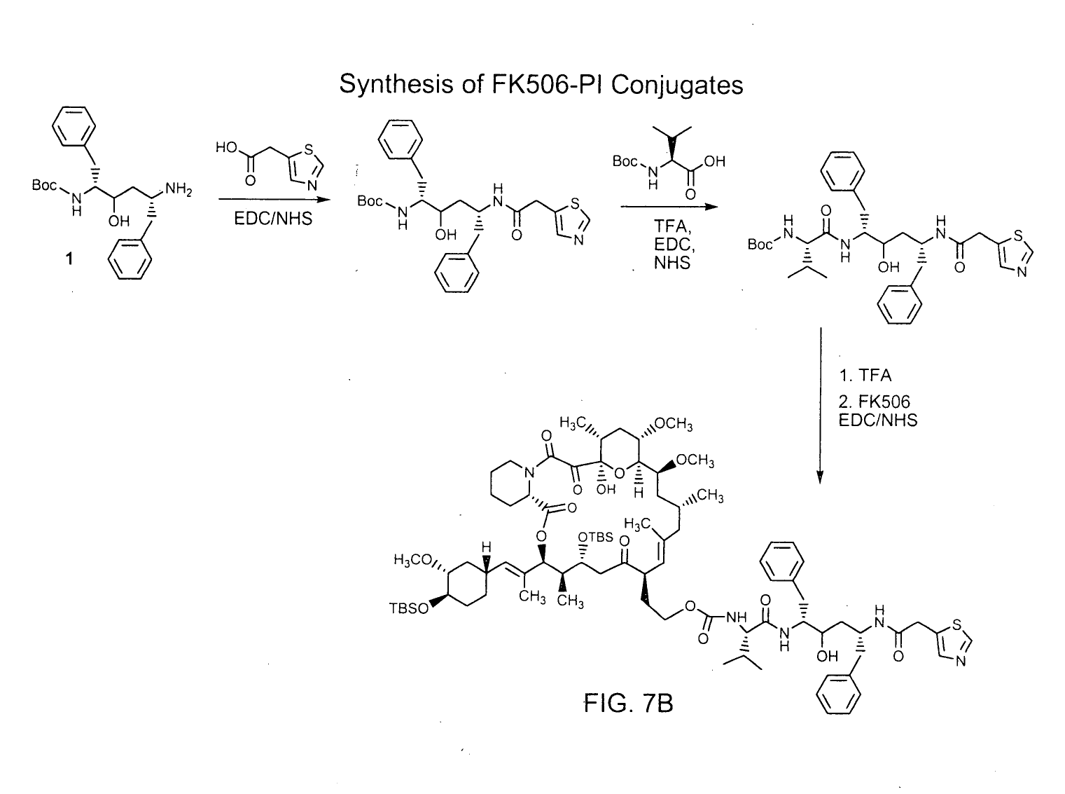

The pharmacokinetic modulating moiety may be, for example, a derivative of FK506, which has high affinity for the FK506-binding protein (FKBP), as depicted for example in FIG. 1. The abundance of FKBP (millimolar) in blood compartments, such as red blood cells and lymphocytes, makes it likely that a significant proportion of a dose of bifunctional compounds comprising FK506 would wind up bound in the blood. A mechanism that tends to increase the portion of the protease dose that winds up in red blood cells and CD4+ lymphocytes will have a favorable effect on anti-HIV activity, as these sites are prime targets of HIV infection and viral load. The steric bulk conferred by FKBP would hinder a protease inhibitor moiety from fitting into the binding pocket of CYP450 enzymes and so would prevent degradation via this class of enzymes.

An inactive form of FK506 may be preferable in some applications to avoid the possibility of side effects due to the possible interaction of the active FK506-FKBP complex with calcineurin. it may be advantageous to use FKBP binding molecules such as synthetic ligands for FKBP (SLFs) described by Holt et al., supra. This class of molecule is lower molecular weight than FK506, and that is generally advantageous for drug delivery and pharmacokinetics. For illustrative purposes, the diagrams will show examples of the use of FK506, though it should be understood that the same strategy can apply to other ligands of peptidyl prolyl isomerases such as the FKBP proteins.

The value of FK506 and other FKBP binding moieties as pharmacokinetic modulating moieties of the invention is further supported by the following. FK506 (tacrolimus) is an FDA-approved immunosuppressant. It has been determined that FK506 can be readily modified such that it loses all immunomodulatory activity but retains high affinity for FKBP. FKBP is an abundant chaperone that is particularly prevalent (˜millimolar) in red blood cells (rbcs) and lymphocytes. The complex between FK506-FKBP gains affinity for calcineurin and inactivation of calcineurin blocks lymphocyte activation and causes immunosuppression.

This interesting mechanism of action is derived from FK506's chemical structure. FK506 is bifunctional; it has two non-overlapping protein-binding faces. One side binds FKBP, while the other binds calcineurin. This property provides FK506 with remarkable specificity and potency. Moreover, FK506 has a long half-life in non-transplant patients (21 hrs) and excellent pharmacological profile. In part, this is because FK506 is unavailable to metabolic enzymes via its high affinity for FKBP, which favors distribution into protected cellular compartments (72-98% in the blood). It can be expected that suitable bifunctional compounds with an FKBP-binding pharmacokinetic modulating moiety will likewise possess some favorable characteristics of inactive FK506, namely, good pharmacokinetics and blood cell distribution.

In general, the pharmacokinetic modulating moiety will have a molecular weight less than about 2000 D, less than about 1600 D, less than about 1300 D, less than about 1100 D, or less than about 900 D.

It is also possible to coadminister the common protein to which the pharmacokinetic modulating moiety binds with the bifunctional compound in order to modify the pharmacokinetics to a greater degree than would be possible with just the native concentration of the common protein.

In a further embodiment of this invention, a method is provided for synthesizing a bifunctional compound comprising protease inhibitor functionality and the ability to bind to a common protein.

The synthesis of the bifunctional compound starts with a choice of suitable pharmacokinetic modulating and drug moieties. It is desirable to identify on each of these moieties a suitable attachment point which will not result in a loss of biological function for either one. This is preferably done based on the existing knowledge of what modifications result or do not result in a biological function. On that basis, it may reliably be conjectured that certain attachment points on the pharmacokinetic and drug moieties do not affect biological function. Thus, for example, in FK506 there is a carboxylic acid function which is suitable as an attachment point. Likewise, in FIG. 4 one sees secondary amine functions on three protease inhibitors which are believed not to significantly affect biological function.

A general synthetic strategy is to locate a secondary amine on the drug moiety at which the drug moiety can be split (so that the secondary amine does not form part of any cycle in the drug moiety). The secondary amine is chosen such that, from experimental or other considerations, it is believed that the drug will retain its efficacy if only the portion of the drug moiety to one side of the secondary amine is present. The portion of the drug moiety to that side of the secondary amine is then synthesized by any appropriate technique, with the secondary amine in the synthesized molecule being protected during synthesis by an appropriate protecting moiety such as Boc. The protecting moiety is then removed, leaving a primary amine which may react with a carboxyl group through a variety of known chemistries for making a peptide bond (see, e.g., J. Mann et al., Natural Products: Their Chemistry and Biological Significance (1994), chapter 3).

In a further aspect of the invention, a bifunctional compound comprising a protease inhibitor moiety with antiviral activity is formulated, for example in the form of a tablet, capsule, parenteral formulation, to make a pharmaceutical preparation The pharmaceutical preparation may be employed in a method of treating a patient having a viral infection against which the protease inhibitor moiety is effective. For example, if the protease inhibitor moiety is effective against the HIV virus, the pharmaceutical preparation may be administered to a patient infected with the HIV virus.

For the preparation of a pharmaceutical formulation containing bifunctional compounds as described in this application, a number of well known techniques may be employed as described for example in Remington: The Science and Practice of Pharmacy, Nineteenth Ed. (Easton, Pa.: Mack Publishing Company, 1995).

In a further aspect of the invention, a bifunctional compound is formulated as part of a controlled release formulation in which an additional controlled release mechanism besides the effect of the pharmacokinetic modulating moiety is employed to achieve desirable release characteristics. The bifunctional compound is as above, comprising a drug moiety, a linker, and a pharmacokinetic modulating moiety. In this aspect of the invention, a drug moiety may be a protease inhibitor or a different type of drug.

As discussed above, bifunctional compounds have the advantage that they can favorably improve the pharmacokinetic characteristics of existing or new drugs. However, in spite of this important advantage, bifunctional compounds used in conventional dosage formulations may not provide enough of an advantage over the mono-functional drug molecules to overcome the additional expense of regulatory approval, drug manufacture, and cost incurred to make the more complex bifunctional compound.

Many mono-functional drugs are not amenable to controlled release technologies due to: 1) short biological half life, 2) potent drug with narrow therapeutic index, 3) require large doses, 4) poor adsorption, 5) poor targeting, 6) low solubility, 7) extensive first-pass metabolism, 8) active adsorption, 9) the time course of the circulating drug does not agree with pharmacological response.

Since the addition of a protein targeting moiety may alter the half life, first-pass metabolism, solubility, targeting, and other properties of monofunctional drugs, the bifunctional strategy can greatly expand the number of drugs which can be used in a controlled release formulation. At the same time, a controlled release formulation of the bifunctional compound can provide enough additional value that may overcome the disadvantage of the extra cost of obtaining regulatory approval for and manufacturing bifunctional drugs compared to mono-functional drugs.

By combining controlled release technologies with bifunctional drugs, it may be possible to 1) enable the use of a wider repertoire of monofunctional drugs in controlled release formats by the addition of a targeting moiety; 2) enhance the value of bifunctional molecules by lowering the required dose and increasing the efficacy of the bifunctional compound relative to a free drug control; 3) alter the pharmacokinetic properties of mixtures of drugs where one or both drugs in the mixture is bifunctional.

For example, in a mixture of methotrexate and clomethiazole, it may be useful to increase the first-pass metabolism of methotrexate using a conjugate to a pharmacokinetic altering moiety such as FK506 or other protein or nucleic acid binding ligand. However, it may be undesirable to alter the pharmacokinetics of clomethiazole where reducing drug clearance might lead to toxic side effects if the patient's liver function is damaged.

In principle, almost any controlled release technology may be applied to a bifunctional compound such as one containing a protease inhibitor. A wide variety of controlled release technologies are known. See, e.g., Encyclopedia of controlled drug delivery (Edith Mathiowitz ed., 1999), and Modern Pharmaceutics (Gilbert S. Banker & Christopher T. Rhodes eds., 4th ed. 2002), especially chapter 15. Exemplary methods of controlled drug delivery include slow erosion, erosion core, pellets in capsules, pellets in tablets, leaching, ion-exchange resins, complexation, microencapsulation, flotation-diffusion, and osmotic pumps.

The drugs that are candidates for bifunctionalization and then the application of other controlled release technologies will generally be those in which other controlled release technologies by themselves do not produce a satisfactory release profile, and bifunctionalization is both possible (e.g., there are suitable linkage points in the drug which do not affect function) and yet does not produce a release profile which is fully adequate.

Drugs which are candidates for bifunctionalization followed by application of other controlled release technologies may belong to a wide variety of therapeutic categories including, but not limited to: analeptic agents; analgesic agents; anesthetic agents; antiarthritic agents; respiratory drugs, including antiasthmatic agents; anticancer agents, including antineoplastic drugs; anticholinergics; anticonvulsants; antidepressants; antidiabetic agents; antidiarrheals; antihelminthics; antihistamines; antihyperlipidemic agents; antihypertensive agents; anti-infective agents such as antibiotics and antiviral agents; antiinflammatory agents; antimigraine preparations; antinauseants; antiparkinsonism drugs; antipruritics; antipsychotics; antipyretics; antispasmodics; antitubercular agents; antiulcer agents; antiviral agents; anxiolytics; appetite suppressants; attention deficit disorder (ADD) and attention deficit hyperactivity disorder (ADHD) drugs; cardiovascular preparations including calcium channel blockers, antianginal agents, central nervous system (CNS) agents, beta-blockers and antiarrhythmic agents; central nervous system stimulants; cough and cold preparations, including decongestants; diuretics; genetic materials; herbal remedies; hormonolytics; hypnotics; hypoglycemic agents; immunosuppressive agents; leukotriene inhibitors; mitotic inhibitors; muscle relaxants; narcotic antagonists; nicotine; nutritional agents, such as vitamins, essential amino acids and fatty acids; ophthalmic drugs such as antiglaucoma agents; parasympatholytics; peptide drugs; psychostimulants; sedatives; steroids, including progestogens, estrogens, corticosteroids, androgens and anabolic agents; smoking cessation agents; sympathomimetics; tranquilizers; and vasodilators including general coronary, peripheral and cerebral.

Exemplary drugs presently known to have high first-pass metabolism include HIV protease inhibitors as discussed above, paclitaxel, methotrexate, vinblastine, verapamil, morphine, lidocaine, acebutolol, isoproterenol, and desipramine. The formation of bifunctional compounds is particularly appropriate for these drugs.

It is to be understood that while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

### Experimental

The general method for testing P1-FK506 conjugates is to synthesize the bifunctional compound and test whether the bifunctional version maintains activity against HIV protease. Then, the P450 susceptibility of the bifunctional compound was tested in a series of fluorescence-based assays. An important aspect of these experiments was the addition of FKBP sources, such as recombinant protein, red blood cells or lymphocytes. The desired outcome is prolonged drug lifetime in the presence of an FKBP source combined with potent anti-HIV protease activity. The synthetic schemes and methods for determining drug lifetime in the presence of P450s will be discussed.

The FDA-approved PIs amprenavir, lopinavir and ritonavir are models for generating FK506-coupled derivatives. These drugs are chosen based on their known defects in pharmacological characteristics. Like all FDA-approved PIs, these compounds are based on peptide substrate sequences. Therefore, we developed a modular amide-based approach to the synthesis of bifunctional compounds (see below). Specifically, an amine in the P2 region (shaded boxes, FIG. 4) is targeted for attachment to a pendant carboxylate on FK506. This region is selected based on structure activity relationships that show the P2 site as a region in which modifications are permitted without affecting biological activity. This approach is amenable to high throughput, solid-phase synthesis if large-scale diversification becomes necessary. These conjugates between FK506 and a PI-derivative are expected to have better activity than the FDA-approved drug on which they are based.

Another feature of the synthesis is that linker bearing an amine on one end and a protected carboxylate on the other can be readily installed as shown in FIG. 6. This capability is designed to allow facile synthesis of analogs with a variety of linker properties (length, flexibility, solubility, etc). For example, longer linkers can be installed if a shorter linker reduces protease inhibition.

### Example 1

### FKBP Protection of Curcumin Conjugates

An amyloid ligand, curcumin, is known to be a good substrate for CYP3a4 (a common P450 enzyme). We investigated whether conjugates between curcumin and FK506 would also be substrates for the enzyme. To test this possibility, we utilized a well-known fluorescence-based CYP3a4 assay. This assay, marketed by Invitrogen (Carlsbad, Calif.), under the name VIVID probes, relies on cytochrome-mediated production of a fluorescent marker from a model substrate. When a compound, such as curcumin, binds to the P450, it displaces the substrate and reduces the rate of production of the fluorescent product. When we tested curcumin-FK506 conjugates in this assay, we found that both curcumin and the conjugate were good substrates for the enzyme. Thus, attachment of FK506 did not appear to significantly alter curcumin's susceptibility to degradation by CYP3a4. However, when we supplied a source of human FKBP (in this case, recombinant bacterially-expressed protein), we observed very different results as shown in FIG. 3. In the presence of FKBP, the curcumin-FK506 conjugate is protected from degradation. FKBP was unable to protect unmodified curcumin, which suggests that the ability to bind FKBP is required for FKBP to have a protective effect. It is believed that FKBP protects the curcumin conjugates from degradation by sterically hindering CYP3a4 binding. In the presence of cellular FKBP sources, this effect would be predicted to be increased because the compartmentalization of the conjugate further reduces availability to P450 enzymes.

### Example 2

### Synthesis of Amprenavir Conjugate

The synthesis of a conjugate based on amprenavir proceeded as outlined below. FIG. 6 depicts the overall synthesis. Briefly, a commercially available Phe-derived epoxide is opened with a valine isostere. The resulting compound is coupled to Boc-protected aminobenzenesulfonyl chloride. Deprotection of the Boc groups is followed by coupling to an activated acid derivative of SLF using 1-ethyl-3-(3-dimethylaminopropyl) carbodiimide hydrochloride (EDC) and N-hydroxylsuccinimide (NHS) (10 equivalents EDC to 1 equivalent NHS). The coupling takes place in dimethylformamide (DMF) at room temperature over four hours. Relative nucleophilicity of the two amines is used to direct amide formation; the benzyl amino group is believed to have diffuse electron density and lowered reactivity. We carried out water work-up and flash chromatography on silica gel using 1:1 ethyl acetate:MeOH. Overall yield was very roughly 15%.

The linkers shown in FIG. 5 may be coupled to FK506 or SLF via EDC-mediated amide formation followed by deprotection of the newly installed carboxylate. This acid is then used for conjugation to the amprenavir-based molecule as above. The linker can be readily altered to enhance solubility or other physical characteristics of the bifunctional compound.

The amprenavir conjugate of this example may also be regarded as a TMC114 conjugate, because TMC114 shares with amprenavir the structure to the right of the attachment point used in this example.

### EXAMPLES 3-4

### Synthesis of Lopinavir and Ritonavir Conjugates

The syntheses of two additional P1-FK506 conjugates may proceed in a fashion generally similar to that employed for the amprenavir-based molecule, as shown in FIGS. 7A-7B. An advanced “Phe-Phe” intermediate 1 can serve as a common precursor for both the lopinavir- and ritonavir-based compounds. Amide formation with one of two carboxylates provides the branch point for the two schemes. In both cases, Boc deprotection provides a handle for creation of the amide with FK506. Various linkers of FIG. 5 may be employed to provide additional diversity and desirable characteristics. Because the linkers are installed on the FK506 moiety, a common pool of FK506-linker molecules may be used on all three synthetic schemes.

### Example 5

### Test of Efficacy of Bifunctional Compounds Against HIV Protease

To analyze efficacy of the conjugates of Example 2 against HIV protease, a commercial biochemical assay was used. The AnaSpec (www.anaspec.com) 71126 HIV-1 protease assay was used according to the manufacturer's direction, except that 1 μM recombinant FKBP was used in some wells. The assay uses a quenched fluorophore substrate. Proteolytic cleavage of the substrate reverses the quench and releases a fluorescent substrate. Experiments were performed in 96-well black Costar plates in real time using a Molecular Devices M5 plate reader. In all cases, fluorescence corrections were made for all drugs and proteins. Linear portions of the curve were used to predict drug lifetime and relative Ki values.

The AnaSpec assay kit shows amprenavir bifunctional retains inhibitory activity against HIV protease. It is twice as effective as the monofunctional TMC114, showing that linker choice has resulted in optimized activity of bifunctional relative to monofunctional.

Similarly, a commercial Invitrogen P2856 assay was used to test for degradation via CYP3a4 in accordance with the manufacturer's directions.

In the presence of 1 μM FKBP, the bifunctional is completely protected from degradation via the CYP3a4. The monofunctional TMC114 compound is >70% degraded under the same condition. In the absence of FKBP, the bifunctional is over 70% degraded by the CYP450 enzyme.

1. A method for modulating at least one pharmacokinetic property of a protease inhibitor upon administration to a host, the method comprising:
administering to the host an effective amount of a bifunctional compound of less than about 5000 daltons comprising the protease inhibitor or an active derivative, fragment or analog thereof and a pharmacokinetic modulating moiety,
wherein the pharmacokinetic modulating moiety binds to at least one intracellular protein and wherein the bifunctional compound has at least one modulated pharmacokinetic property upon administration to the host as compared to a free drug control that comprises the protease inhibitor.

2. The method according to claim 1, wherein the pharmacokinetic property is selected from the group consisting of half-life, hepatic first-pass metabolism, volume of distribution, and degree of blood protein binding.

3. The method according to claim 1, wherein the bifunctional compound is administered as a pharmaceutical preparation.

4. The method according to claim 1, wherein the host is a mammal.

5. The method according to claim 1, wherein the pharmacokinetic property is half-life.

6. The method of claim 1, wherein the pharmacokinetic property is hepatic first-pass metabolism.

7. The method of claim 1, wherein the at least one intracellular protein bound comprises an FK506 binding protein, tubulin, actin, a heat shock protein, or albumin.

8. The method of claim 1, wherein the at least one intracellular protein bound comprises an FK506 binding protein.

9. The method of claim 1, wherein the pharmacokinetic modulating moiety has a molecular weight less than about 1100 daltons.

10. A bifunctional compound comprising a protease inhibitor moiety, a linker, and a pharmacokinetic modulating moiety, wherein the linker is attached to the protease inhibitor moiety and the pharmacokinetic modulating moiety, and the pharmacokinetic modulating moiety gives the bifunctional compound a different pharmacokinetic behavior from that of the protease inhibitor moiety in the absence of the linker and pharmacokinetic modulating moiety, wherein the molecular weight of the bifunctional compound is less than about 5000 daltons and the molecular weight of the pharmacokinetic modulating moiety is less than about 1100 daltons.

11. The bifunctional compound according to claim 10 where the attachment point of the linker to the protease inhibitor moiety has been optimized for best drug activity relative to other bifunctional compounds having the same protease inhibitor moiety and pharmacokinetic modulating moiety.

12. The bifunctional compound according to claim 10 where the attachment point of the linker to the pharmacokinetic modulating moiety has been optimized for best drug activity relative to other bifunctional compounds having the same protease inhibitor moiety and pharmacokinetic modulating moiety.

13. The bifunctional compound according to claim 10 where the attachment point of the linker to the pharmacokinetic modulating moiety has been optimized to improve pharmacokinetics relative to other bifunctional compounds having the same protease inhibitor moiety and pharmacokinetic modulating moiety.

14. A bifunctional compound according to claim 10, where the linker comprises at least three carbons.

15. A bifunctional compound according to claim 10, where the efficacy of the bifunctional compound in the presence of a suitable protein to which the pharmacokinetic modulating moiety couples is increased relative to the efficacy of the protease inhibitor moiety.

16. A bifunctional compound according to claim 15, where the efficacy of the bifunctional compound in the presence of a suitable protein to which the pharmacokinetic modulating moiety couples is increased by a factor of at least about 2 relative to the efficacy of the protease inhibitor moiety.

17. In a method of administering a drug to a host in need of said drug, the improvement comprising:
administering to said host an effective amount of a bifunctional compound comprising said drug or a derivative, fragment or analog thereof linked to a ligand for a presenter protein endogenous to said host, wherein said drug binds to a drug target and said ligand binds to a presenter protein that is not said drug target, wherein the bifunctional compound is administered in a controlled release formulation that operates according to a controlled release mechanism in addition to whatever controlled release is provided by the ligand.

18. The method according to claim 17 where the bifunctional compound has a molecular weight of less than 5000 daltons.

19. The method according to claim 17 where the presenter protein ligand has a molecular weight of less than 5000 daltons.

20. The method of claim 17, wherein the drug is a protease inhibitor.

21. The method according to claim 17, wherein the host is a mammalian host.

22. The method according to claim 17, wherein the mammalian host is human.

23. The method of claim 17 where the presenter protein ligand is FK506.

24. The method of claim 17 where the presenter protein ligand target is a peptidyl prolyl isomerase.

25. The method of claim 17 where the presenter protein ligand targets an intracellular protein.

26. The method of claim 17 where the presenter protein ligand binds to a derivative of cyclosporin.

27. The method of claim 17 where the presenter protein ligand binds to FKBP.

28. The method of claim 17 where the presenter protein ligand binds to albumin.

29. The method according to claim 17, wherein the controlled release mechanism is slow erosion.

30. The method according to claim 17, wherein the controlled release mechanism is erosion core only.

31. The method according to claim 17, wherein the controlled release mechanism is pellets in capsules.

32. The method according to claim 17, wherein the controlled release mechanism is pellets in tablets.

33. The method according to claim 17, wherein the controlled release mechanism is leaching.

34. The method according to claim 17, wherein the controlled release mechanism is ion-exchange resins.

35. The method according to claim 17, wherein the controlled release mechanism is complexation.

36. The method according to claim 17, wherein the controlled release mechanism is microencapsulation.

37. The method according to claim 17, wherein the controlled release mechanism is flotation-diffusion.

38. The method according to claim 17, wherein the controlled release mechanism is an osmotic pump.

39. A method for modulating at least one pharmacokinetic property of a drug upon administration to a host, the method comprising:
administering to the host an effective amount of a bifunctional compound of less than about 5000 daltons comprising the drug or an active fragment, analog, or derivative thereof and a pharmacokinetic modulating moiety, wherein the pharmacokinetic modulating moiety binds to at least one intracellular protein and wherein the bifunctional compound modulates at least one pharmacokinetic property and one efficacy property upon administration to the host as compared to a free drug control.
